(19) United States
(12) Patent Application Publication
(10) Pub. No.: US 2025/0302390 A1
(43) Pub. Date: Oct. 2, 2025

(54) MONITORING AND DETECTING VITAL SIGN DECOMPOSITION

(71) Applicant: KONINKLIJKE PHILIPS N.V., EINDHOVEN (NL)

(72) Inventors: Sjoerd Herman Garssen, Eindhoven (NL); Wilhelmus Franciscus Johannes Verhaegh, Eindhoven (NL)

(21) Appl. No.: 19/096,877

(22) Filed: Apr. 1, 2025

Related U.S. Application Data
(60) Provisional application No. 63/572,538, filed on Apr. 1, 2024.

(51) Int. Cl. A61B 5/00; A61B 5/0205

(52) U.S. Cl. CPC A61B 5/7228; A61B 5/0205; A61B 5/4842; A61B 5/7264

(57) ABSTRACT

A method for identifying deterioration of a patient's condition, comprising: obtaining, by a wearable vital sign sensor from the patient during a first time period, vital sign data; decomposing the vital sign data into one or more components, comprising: (i) a trend component characterizing a mean of the vital sign data over the first time period; (ii) a periodic component characterizing periodicity of the vital sign data over the first time period; and (iii) a residual component over the first time period; analyzing the one or more components to determine whether there is deterioration in the patient's condition; identifying, based on the analysis, a deterioration in the patient's condition, wherein the deterioration is a current deterioration or an impending deterioration; and reporting the identification of the deterioration in the patient's condition.

### BACKGROUND OF THE INVENTION

### 1. Field of the Invention

The present disclosure is directed generally to methods and systems for identifying deterioration of a patient's condition using vital sign data obtained with a wearable device.

### 2. Description of the Related Art

Healthcare is challenged by an aging population and shortages of hospital beds, among many other challenges. A potential solution can be found in electronic Health (eHealth, which includes remote patient monitoring. This can be utilized, for example, to transfer patient care from hospitals to the patients' own homes, which has shown promising results. However, for safety reasons, it is important that deterioration of remotely monitored patients is recognized at the earliest stage possible. Thus, there is a need for the early prediction of whether remotely monitored patients have a high chance of deterioration, or are experiencing deterioration.

Deterioration prediction for remotely monitored patients has received some scientific attention. For example, the Remote Early Warning Score (REWS) has shown to be potentially useful. The REWS uses a score-distribution scheme that is similar to a traditional Early Warning Score (EWS), except that only heart rate (HR) and respiratory rate (RR) are taken into account. However, its performance has only been tested on a small group of oncological patients. As another example, some research has been done for the prediction of deterioration of COPD and asthma patients, although the prediction models are not yet ready to be clinically useful.

Since the use of wearable sensor is on the rise, the vital signs of remote patients are increasingly being measured in a continuous way. Since vital signs often show precedent signals before deterioration occurs, this data is likely to be useful for predicting remote patient deterioration. However, to use this data, some challenges still need to be solved. For example, one challenge is the processing of remote continuous vital sign data. Raw data could be used, as is done in Early Warning Score (EWS) systems. However, when monitoring continuously, many variations in vital signs occur that do not have a clinical meaning. Hence, it would be beneficial to separate meaningful variations from other variations.

### SUMMARY OF THE INVENTION

There is thus a continued unmet need for methods and systems that efficiently and accurately identify deterioration of a remote patient's condition using vital sign data.

Various embodiments and implementations are directed to a method and system for identifying deterioration of a remote patient's condition, using deterioration detection system. The deterioration detection system obtains, by a wearable vital sign sensor from the patient during a first time period, vital sign data. The system then decomposes that vital sign data into at least three components, comprising: (i) a trend component characterizing a running mean of the vital sign data over the first time period; (ii) a periodic component characterizing periodicity of the vital sign data over the first time period; and (iii) a residual component over the first time period. The system analyzes the three components to determine whether there is deterioration in the patient's condition, and identifies a deterioration based on that analysis. The identified deterioration is reported to a clinician via a user interface.

According to an aspect, a method for identifying deterioration of a patient's condition. The method includes: obtaining, by a wearable vital sign sensor from the patient during a first time period, vital sign data: decomposing the vital sign data into one or more components, comprising: (i) a trend component characterizing a mean of the vital sign data over the first time period: (ii) a periodic component characterizing periodicity of the vital sign data over the first time period; and (iii) a residual component over the first time period: analyzing the one or more components to determine whether there is deterioration in the patient's condition: identifying, based on the analysis, a deterioration in the patient's condition, where the deterioration is a current deterioration or an impending deterioration; and reporting the identification of the deterioration in the patient's condition.

According to an embodiment, the method further includes: obtaining, during a second time period after the first time period, additional vital sign data: decomposing the additional vital sign data into one or more components, comprising: (i) the trend component characterizing a mean of the vital sign data over the second time period: (ii) the periodic component characterizing periodicity of the vital sign data over the second time period; and (iii) the residual component over the second time period: wherein the one or more components over the second time period are analyzed to determine whether there is deterioration in the patient's condition.

According to an embodiment, the method includes transmitting the vital sign data to a remote server.

According to an embodiment, the residual component characterizes variation in the vital sign data, wherein the variation is not explained by the trend component or the periodic component.

According to an embodiment, decomposing the vital sign data into at least three components comprises inflation weighting.

According to an embodiment, the first time period is at least 24 hours, or at least one circadian rhythm.

According to an embodiment, the vital sign data is one or more of heart rate, blood pressure, respiration rate, activity, and posture.

According to an embodiment, one or more of the steps of: (i) analyzing the one or more components to determine whether there is deterioration in the patient's condition; and (ii) identifying, based on the analysis, deterioration in the patient's condition, comprises a machine learning algorithm trained to perform the analysis and/or identification.

According to an embodiment, identifying, based on the analysis, deterioration in the patient's condition comprises a deterioration score, and wherein reporting the identification of the deterioration in the patient's condition comprises the deterioration score.

According to another aspect is a system for identifying deterioration of a patient's condition. The system includes: a wearable device comprising a vital sign sensor configured to obtain vital sign data from a patient: a processor configured to: (i) decompose the vital sign data into one or more components, comprising: (1) a trend component characterizing a mean of the vital sign data over the first time period: (2) a periodic component characterizing periodicity of the vital sign data over the first time period; and (3) a residual component over the first time period: (ii) analyze the one or more components to determine whether there is deterioration in the patient's condition: (iii) identify; based on the analysis, a deterioration in the patient's condition, where the deterioration is a current deterioration or an impending deterioration; and a user interface configured to report the identification of the deterioration in the patient's condition.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a method for identifying deterioration of a patient's condition, in accordance with an embodiment.

FIG. 2 is a schematic representation of a deterioration detection system, in accordance with an embodiment.

FIG. 3 is a schematic representation of a time series decomposition, in accordance with an embodiment.

FIG. 4 is a flowchart of a method for training a deterioration detection model, in accordance with an embodiment.

FIG. 5 is a schematic representation of a method for identifying deterioration of a patient's condition, in accordance with an embodiment.

FIG. 6 is a graph of false alarms following different methods of deterioration detection model training, in accordance with an embodiment.

FIG. 7 is a graph of false alarms following different methods of deterioration detection model training, in accordance with an embodiment.

### DETAILED DESCRIPTION OF EXEMPLARY EMBODIMENTS

The present disclosure describes various embodiments of a system and method configured to identify deterioration of a remote patient's condition, using a deterioration detection system. More generally, Applicant has recognized and appreciated that it would be beneficial to provide a method and system to efficiently and accurately identify deterioration of a remote patient's condition using vital sign data. A deterioration detection system obtains, by a wearable vital sign sensor from the patient during a first time period, vital sign data. The system then decomposes that vital sign data into at least three components, comprising: (i) a trend component characterizing a mean of the vital sign data over the first time period; (ii) a periodic component characterizing periodicity of the vital sign data over the first time period; and (iii) a residual component over the first time period. The system analyzes the three components to determine whether there is deterioration in the patient's condition, and identifies a deterioration based on that analysis. The identified deterioration is reported to a clinician via a user interface.

The embodiments and implementations disclosed or otherwise envisioned herein can be utilized with any system that may utilize or benefit from monitoring a remote patient's vital sign data. For example, one application of the embodiments and implementations disclosed or otherwise envisioned herein is patient monitoring using vital sign data obtained by a wearable device. However, the disclosure is not limited to these devices or systems, and thus disclosure and embodiments disclosed herein can encompass any system that may utilize or benefit from monitoring a remote patient's vital sign data.

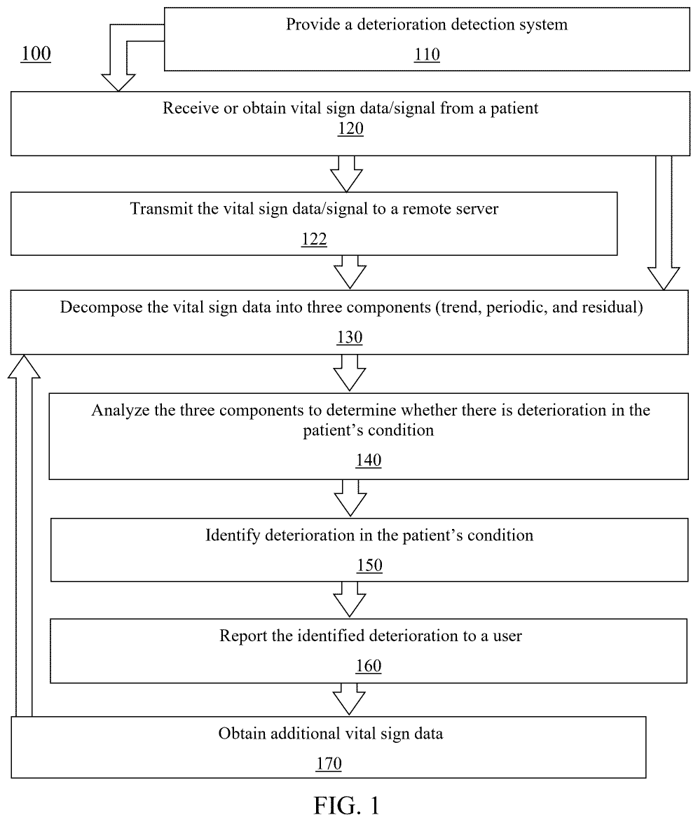

Referring to FIG. 1, in one embodiment, is a flowchart of a method 100 for identifying deterioration of a patient's condition using a deterioration detection system. The methods described in connection with the figures are provided as examples only, and shall be understood not to limit the scope of the disclosure. The deterioration detection system can be any of the systems described or otherwise envisioned herein. The deterioration detection system can be a single system or multiple different systems.

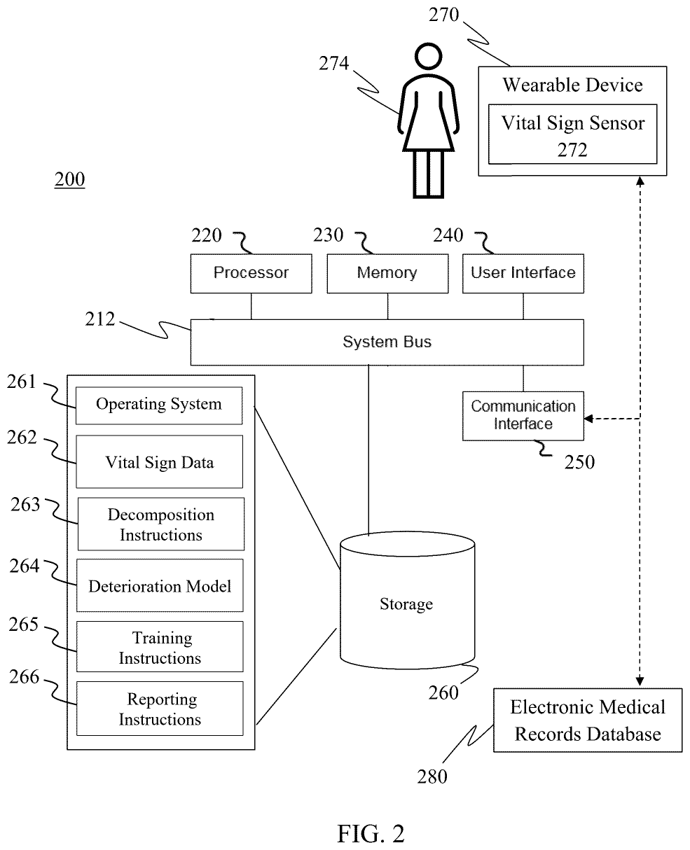

At step 110 of the method, a deterioration detection system 200 is provided. Referring to an embodiment of a deterioration detection system 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the system 200 may be different and more complex than illustrated. Additionally, deterioration detection system 200 can be any of the systems described or otherwise envisioned herein. Other elements and components of the deterioration detection system 200 are disclosed and/or envisioned elsewhere herein.

According to an embodiment, the deterioration detection system 200 comprises or is in direct or indirect communication with a wearable device 270. The wearable device is any device that is in direct or indirect communication with a patient 274. For example, the wearable device may be a device designed to be worn by the patient, such as on the arm, around the neck, on or around the face, or anywhere else on the patient's body. The wearable device may be clipped or connected to the patient's clothing. The wearable device may be in proximity to the patient without direct contact. For example, the wearable device may be near the patient and able to obtain vital sign data via a mechanism such as a camera, sound sensor, and/or via any other mechanism for obtaining vital sign information. Many other forms of the wearable device are possible.

According to an embodiment, the wearable device 270 comprises a vital sign sensor 272. The vital sign sensor is any sensor configured or otherwise capable of obtaining a vital sign signal from the patient 274. The vital sign may be any vital sign obtainable from a patient, including but not limited to body temperature, pulse or heart rate, respiration rate, blood pressure, and more. Thus, according to an embodiment, the vital sign sensor 272 is or comprises a thermometer, a pulse oximeter (for photoplethysmography), an acoustic-based sensor, and/or an inductance plethysmography, capnography, piezoelectric, accelerometer, or bioimpedance-based sensor, among other possible sensors. The wearable device 270 can receive the vital sign signal obtained from the patient 274 by the vital sign sensor 272 in real-time, or can receive it periodically or in response to a request for data. Once received, the vital sign signal may be utilized immediately and/or may be stored in local and/or remote memory.

According to an embodiment, the deterioration detection system 200 comprises or is in direct or indirect communication with an electronic medical record system and/or an electronic medical records (EMR) database 280 from which the information about a patient, including vital sign data, demographic information, diagnosis information, and/or treatment information, may be obtained or received. For example, EMR database may comprise historical vital sign data about the patient, among other patient information. According to an embodiment, the electronic medical record system 280 may be a local or remote database and is in direct and/or indirect communication with system 200. Thus, according to an embodiment, the system comprises an electronic medical record database or system 280.

At step 120 of the method, the deterioration detection system 200 obtains or receives vital sign data from patient 274 via the vital sign sensor 272 of wearable device 270. This vital sign data or signal can be any of the vital sign data described or otherwise envisioned herein, such as body temperature, pulse or heart rate, respiration rate, blood pressure, activity data, and posture data, among other possible vital sign data. The deterioration detection system 200 can receive the vital sign signal from the patient in real-time, or can receive it periodically or in response to a request for data. For example, the vital sign sensor, wearable device, and/or deterioration detection system can be designed or programmed to provide vital sign data in real-time, periodically pursuant to a predetermined schedule or trigger(s), and/or in response to a request for vital sign data from a user, a clinician, and/or from another component of the deterioration detection system. According to another embodiment, some or all of the vital sign data is received from the EMR database 280. Once received, the vital sign signal may be utilized immediately and/or may be stored in local and/or remote memory.

At optional step 122 of the method, the obtained or received vital sign data is transmitted via a wired and/or wireless communication network to a remote server. According to an embodiment, the deterioration detection system comprises a remote server or processor (among other possibly remote components) that performs one or more downstream steps of the method. Accordingly, in this embodiment, the wearable device is configured to obtain and transmit the vital sign data, and one or more remote servers or processors are configured to receive and utilize that transmitted vital sign data. The remote server or processor can be located in proximity to the patient, such as the patient's smartphone, computer, or other device. Alternatively; the remote server or processor can be located remote to the patient, such as a server or processor located at a centralized location configured to monitor and analyze data for a plurality of patients. According to an embodiment, analysis of the received vital sign data can be a service to which the patient or the healthcare professional is subscribed or otherwise engaged with.

At step 130 of the method, the deterioration detection system 200 decomposes the vital sign data into one or more components. According to an embodiment, in a time series analysis, a signal can often be decomposed into at least three components. The first component is a trend component, which describes how the mean of the signal behaves over time. The second component is a periodic or seasonality component, which describes the periodicity of the time series, which for vital signs is typically a 24-hour circadian rhythm although the time can vary. The third component is a residual component that exists due to random noise or an unknown cause, which may be an unnoticed deterioration. One advantage of this decomposition approach is that it yields or characterizes information on the circadian rhythm that may be relevant for deterioration detection, and also yields or characterizes other variations when they are placed into a more appropriate context. For example, a heart rate of 90 may be perfectly fine during the day, but could be exceptionally high during the night when a patient is sleeping.

According to an embodiment, in order to decompose the vital sign time series, an initialization period of at least one time period is needed. In case of vital signs this may be about 24 hours, which is the length of a single circadian rhythm or cycle. According to an embodiment, this initialization period may already be available if a patient receives a wearable sensor during a hospital stay and the sensor stays attached to the patient after hospital discharge to monitor the patient remotely. In this case, it is important to note that circadian rhythms may differ between a patient being at home or in the hospital, e.g., due to earlier breakfasts in the hospital and many other causes. Thus, when a patient is at home, it may be preferred to use data generated at home for the decomposition. However, in the first day(s) after discharge, there is limited data yet generated at home. Therefore, the decomposition will be largely based on data that was generated during hospital stay, as there is not enough home data to decompose the vital sign time series. But when the patient is at home for several days, the effect of the data that was generated during hospital on the time series decomposition should vanish.

Accordingly, decomposition of the vital sign signal into the components is based on exponential smoothing, such that the longer ago the data was measured, the less weight it has in the extraction of the periodic/seasonality component. In this way, directly after hospital discharge, the system is able to extract the components with the best information that is available at that moment. As time proceeds, data generated during hospital stay becomes less important to extract the periodic/seasonality component. Thus, the system is able to use vital sign decomposition as early as possible while the decomposition is increasingly tailored to the home situation of a patient, which improves the quality of the decomposition.

In accordance with an embodiment, with inflation weighting, older data can be weighted with a lower factor than newer data, but the exact formula may still be a choice. Exponentially decaying weights (exponential smoothing) is one solution, but weights might also be scaled down linearly (e.g. data of x days ago may be weighted down by 10x percent). Many other options are possible.

According to an embodiment, therefore, the deterioration detection system decomposes the vital sign data into at least three components, namely the trend component, the periodic component, and the residual component. When decomposing a signal, especially in the context of time series analysis, the residual component represents the portion of the signal that is not explained by the trend and periodic components identified during decomposition. According to an embodiment, the residual component characterizes fluctuations, noise, or anomalies in the data. Thus, analyzing the residual component can help characterize underlying patterns of the data that may otherwise be obfuscated in a non-decomposed signal.

According to an embodiment, the data decomposition requires an initialization period of at least 24 hours, although less or more time for the initialization period is possible. After the initialization period, during which vital sign data is obtained for the patient as described or otherwise envisioned herein, the vital sign time series can be decomposed in the following way for day d and hour h:
1. Resample the obtained vital sign data to hourly values (although smaller and larger time periods are possible); and
2. Calculate the trend by averaging over the preceding 24 hours using Equation 1 (below), in which Raw_{d,h} is the resampled raw data at day d and hour h after step 1, and t is the number of hours going back in time. Notably, a smaller and larger time period than the preceding 24 hours is possible. Thus, according to an embodiment, the trend is generated during the decomposition process using the following equation:

$${Trend}_{d , h} = \frac{1}{24} {\sum }_{t = 0}^{23} {Raw}_{( d , h ) - t} \qquad ( Eq . 1 )$$

3. The detrended time series is then calculated using the following equation:

$$Detr e {nded}_{d , h} = R a {w}_{d , h} - T r e n {d}_{d , h} \qquad ( Eq . 2 )$$

4. From the detrended data, the periodic (e.g., “circadian rhythm”) component is calculated by determining the mean grouped by hour, using exponential smoothing. Exponential smoothing is done using Equation 3 below (with smoothing factor α=0.33, note that this smoothing factor can be chosen differently depending on the aim). In this way, the older an observation is, the less impact it has on the current periodic value.

$$Circadian {rhythm}_{d , h} = \frac{{\sum }_{t = 0}^{d} {( 1 - α )}^{d - t} \cdot {Detrended}_{t , h}}{{\sum }_{t = 0}^{d} {( 1 - α )}^{d - t}} \qquad ( Eq . 3 )$$

5. The periodic component (“circadian rhythm”) is then subtracted from the detrended data to obtain the residual, using the following equation:

$${Residual}_{d , h} = {Detrended}_{d , h} - Circadian {rhythm}_{d , h} \qquad ( Eq . 4 )$$

Thus, following step 5 (and thus following step 130 of method 100 in FIG. 1), the vital sign signal is decomposed into the trend component, the periodic component, and the residual component.

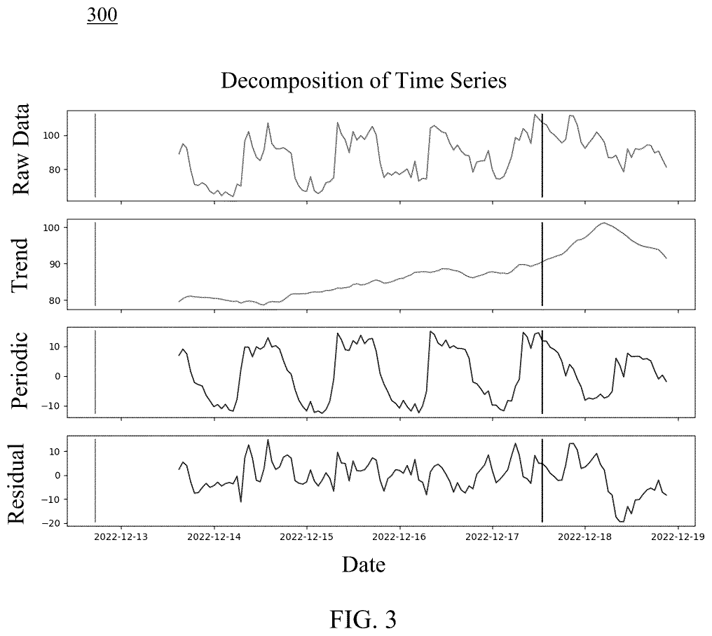

Referring to FIG. 3, in one embodiment, is an example decomposition 300 of a vital sign signal from a patient. In this example, the data is heart rate data from a patient that wore a wearable sensor obtaining the vital sign data. The data comprises raw data (“Raw Data”) which is the vital sign signal obtained from the patient. The data is decomposed into the three components, namely the trend component (“Trend”), the periodic component (“Periodic”), and the residual component (“Residual”). In this example, the bar on the lefthand side is the time of hospital discharge, and the bar on the righthand side is the time of hospital readmission. In accordance with an embodiment, the initial period of vital sign data (such as the previous 24 hours) is not plotted because of a run-in effect, and thus not all components may be available yet.

Returning to method 100 in FIG. 1, at step 140 of the method the deterioration detection system 200 analyzes the one or more components from the decomposition, specifically to determine whether or not there is a deterioration in the patient's condition. The advantage of this novel system is that it can detect deterioration in the patient's condition better than other monitoring systems, since it is analyzing at least the residual component to identify a pattern (such as deterioration or lack of deterioration) in the signal separate from the trend and periodic components or patterns. Indeed, this approach allows the data to be put in context of the trend and periodicity; which significantly improves the resulting analysis.

According to an embodiment, the deterioration detection system 200 analyzes one of the components from the decomposition, whether the decomposition results in one component or multiple components. According to another embodiment, the deterioration detection system analyzes two of the components from the decomposition. According to yet another embodiment, the deterioration detection system analyzes three of the components from the decomposition. And according to another embodiment, the deterioration detection system analyzes three of the components from the decomposition as well as other data, including but not limited to the raw vital sign data. Other variations are possible.

At step 150 of the method, the deterioration detection system 200 identifies a deterioration in the patient's condition based on the analysis in step 140 of the method. For example, the system may identify a deterioration after determining that there is a pattern in the residual component that is associated with a possible or confirmed deterioration.

In accordance with another embodiment, at step 150 of the method, the deterioration detection system 200 predicts an upcoming or impending deterioration in the patient's condition based on the analysis in step 140 of the method. For example, the system may identify a pattern in the analysis, thereby predicting that—based on the pattern—deterioration is upcoming or impending. Based on the pattern and/or on the training of the system, the prediction may include a timeframe or other estimate of time to the upcoming or impending deterioration.

According to an embodiment, the deterioration detection system 200 analyzes one or more of the three components to determine whether or not there is a deterioration in the patient's condition and/or to identify the deterioration, using one or more of a variety of different possible methods. For example, the system can analyze the one or more of the three components using a set of rules or thresholds in order to determine that there is a pattern in the residual component that is associated with a possible or confirmed deterioration.

According to another embodiment, the deterioration detection system 200 utilizes a trained deterioration detection model to analyze one or more of the three components to determine whether or not there is a deterioration in the patient's condition and/or to identify the deterioration. The trained deterioration detection model can be any model that can be trained to utilize the input to generate the output, as described or otherwise envisioned herein. For example, the deterioration detection model can be a neural network or other trained machine learning model. Thus, according to an embodiment, the deterioration detection system 200 comprises a trained deterioration detection model that receives the input data (i.e., one or more of the three components) and outputs an identification of deterioration in the patient's condition. That output can include, for example, a score or other visual, audible, or other indication of deterioration in the patient's condition

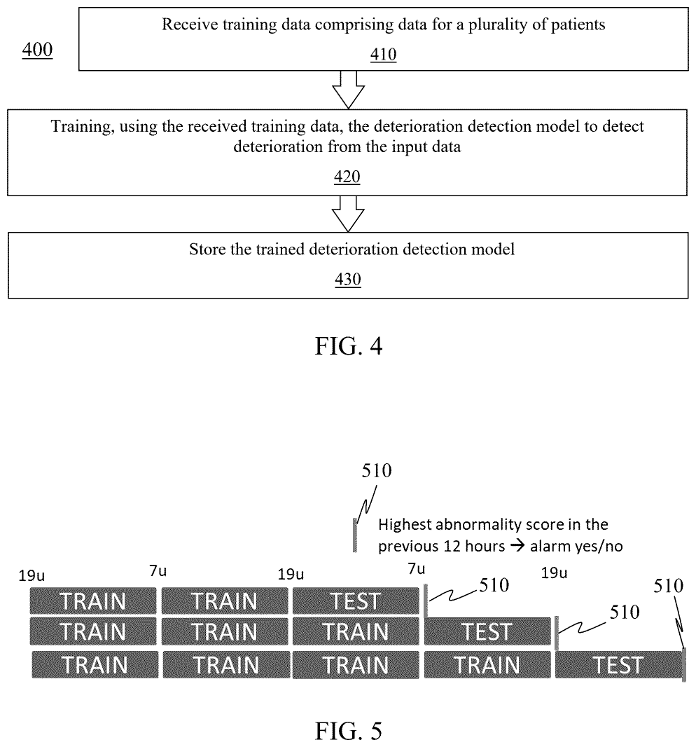

The deterioration detection model can be trained in a variety of different ways. According to one embodiment, the deterioration detection model is trained in a supervised or unsupervised manner, among other possible training methods. Referring to FIG. 4, in one embodiment, is a flowchart of a method 400 for training the deterioration detection model of the deterioration detection system 200. This method may be performed by the deterioration detection system, and/or may be performed by another system such as a specialized machine learning model training system.

At step 410 of the method, the training system receives training data which will be used to train the model. The training data can be any data sufficient to train the model to utilize the described input data to generate the described output. For example, the training data may comprise decomposed components from vital sign data for a plurality of patients, including patients some of which are known to have experienced deterioration and some of which did not experience deterioration, which thus may include ground truth optimization. This training data, which could be utilized in a supervised or unsupervised manner, can comprise raw and/or decomposed vital sign data for 100 s or 1000 s of patients, and can be updated with new data. The training data may also comprise other information. This training data may be obtained and curated by an expert such as a clinician, or it may be obtained and curated under the supervision of a clinician, or it may be obtained and utilized without curation. The training data may be received from any source. For example, the training data may be received from the electronic medical record database or system 280, or any other component of the system or a training system. According to an embodiment, system 200 comprises or is in direct or indirect communication with a database which comprises some or all of the training data set.

According to an embodiment, the decomposed signals optionally together with the original raw signals, can be used to define a number of features that may be utilized for a machine learning approach. For example, referring to TABLE 1 is a noncomprehensive list of possible features.

TABLE 1
Possible engineered features from sensor data.

| Portion or Component | Possible Features |
|---|---|
| Raw | Slope (HR, RR, Act, Lying) |
| | Mean (HR, RR, Act, Lying) |
| | Relative Mean (HR, RR, Act) |
| | Range 24 h (HR, RR, Act) |
| | Peak count 24 h (HR, RR, Act) |
| | Standard deviation (HR, RR, Act) |
| | 10^{th} percentile (HR, RR, Act) |
| | 90^{th} percentile (HR, RR, Act) |
| | Pearson correlation coefficient (HR & Act) |
| Trend | Slope (HR, RR, Act, Lying) |
| | Mean (HR, RR, Act, Lying) |
| | Relative Mean (HR, RR, Act) |
| Circadian rhythm | Range 24 h (HR, RR, Act) |
| | Peak count 24 h (HR, RR, Act) |
| Residual | Mean (HR, RR, Act) |
| | Standard deviation (HR, RR, Act) |
| | 10^{th} percentile (HR, RR, Act) |
| | 90^{th} percentile (HR, RR, Act) |
| | Pearson correlation coefficient (HR & Act) |

According to an embodiment, the Pearson correlation coefficient quantifies the strength of the relationship between activity and HR. In this non-limiting example, all features were calculated over a time window of six hours, which was chosen based on discussions with clinical experts and literature, except for the features aimed to quantify the circadian rhythm (range and peak count). To extract a feature value from a time window, at most 50% of the hours within the time window were allowed to have missing values. The features for the raw dataset were chosen similarly to the features extracted from the decomposed dataset. Relative mean features were calculated as a patient's current mean compared to its mean over the first three hours.

Thus, there are many options for feature extraction, including the features described in detail above with regard to TABLE 1, which are extracted based on time windows. It is also possible to use (hourly) residual values (and/or trend values and/or seasonal values) as input for the prediction model rather than features extracted from time windows. According to another embodiment, the features (such as the features from TABLE 1) can be extracted or determined using sliding windows, such as over a preceding time window. As just one non-limiting example, the preceding time window can be a window of six hours, although shorter or longer windows are possible.

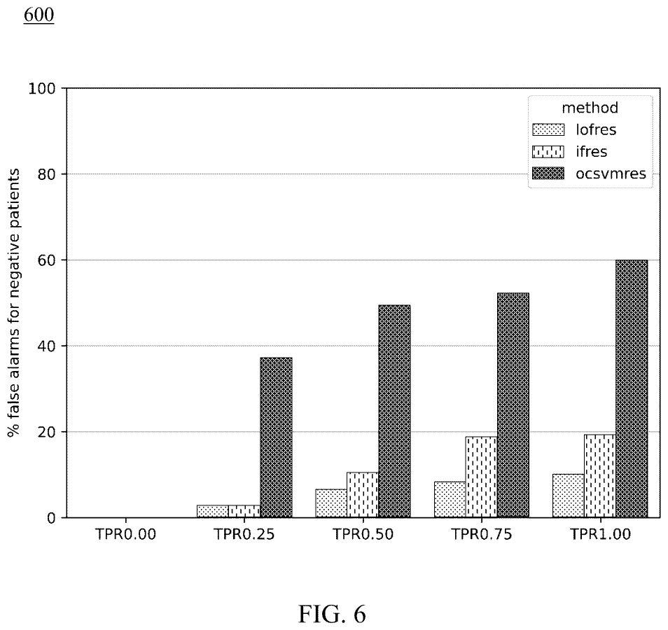

Referring to FIG. 6, in one embodiment, is a graph 600 of results using the residuals of heart rate, respiratory rate, and activity level to predict patient deterioration using patient-based models. The y-axis represents the percentage of all decision-moments for non-deteriorated patients to result in a false alarm, for which there are two decision-moments per day (7 AM and 7 PM). The x-axis shows the true positive rate, corresponding to different cut-off values on the score. Three models were used: local outlier factor using residuals data (“lofres”), isolation forest (“ifres”), and one-class support vector machine (“ocsvmres”). Note that for this experiment, only 4 patients deteriorated, therefore only a few levels of the true positive rate could be shown. The optimal feature(s) may also depend on the use case and patient population. For example, features extracted from the respiratory rate could be useful for patient with a respiratory disease, while for other patients such features might be irrelevant.

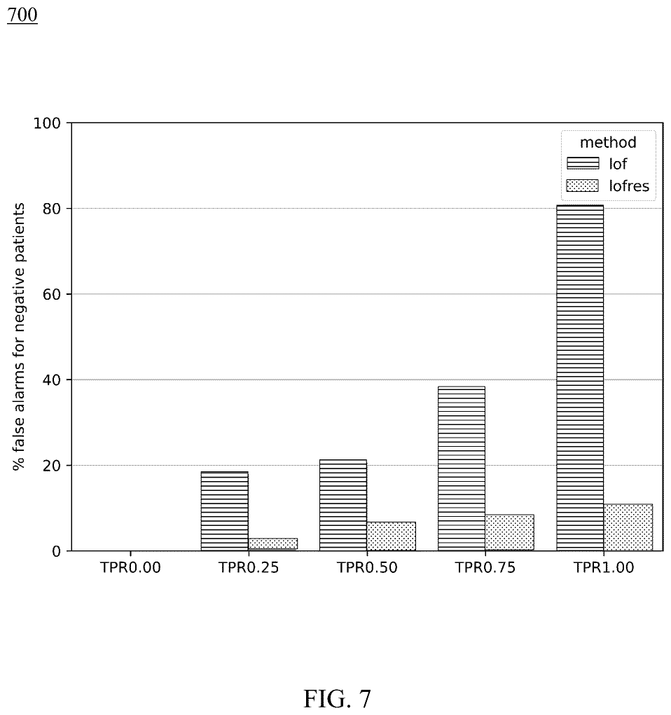

Referring to FIG. 7, in one embodiment, is a graph 700 of results using decomposition components to predict patient deterioration. The y-axis represents the percentage of all decision-moments for non-deteriorated patients to result in a false alarm. The x-axis shows the true positive rate. Two models were used: local outlier factor using residuals data (“lofres”) and local outlier factor using raw vital sign data (“lof”). The data clearly shows that lofres is preferred over lof, meaning that with vital sign data decomposition there are significantly better results.

According to an embodiment, the training system may comprise a data pre-processor or similar component or algorithm configured to process the received training data. For example, the data pre-processor analyzes the training data to remove noise, bias, errors, and other potential issues. The data pre-processor may also analyze the input data to remove low quality data. Many other forms of data pre-processing or data point identification and/or extraction are possible.

At step 420 of the method, the training system trains the deterioration detection model, using the training data, to analyze one or more of the three components (alone and/or with the raw vital sign signal) to determine whether or not there is a deterioration in the patient's condition and/or to identify the deterioration. The deterioration detection model is trained using any method for training such a model. The trained deterioration detection model is a unique model based on the training data used to train the model. Following training, the system comprises a trained deterioration detection model.

According to an embodiment, the features described or envisioned herein (including but not limited to features such as those found in TABLE 1) can be used to train and test the machine learning algorithm. In some embodiments, particularly where there may be a limited number of available home deteriorations to utilize for the training data, it may be more difficult to develop a supervised machine learning model using positive and negative cases. Thus, the system can utilize a one-class classification approach (and in a specific example, a one-class support vector machine), that builds a model only using the negative cases. This leads to a model that can give a score to a new sample, and based on this score one can call the new sample an outlier to the negative class (i.e., likely to be of the positive class) or as a part of the negative class. Many other options are possible.

According to an embodiment, the deterioration detection model can be trained to provide a score as an output of the analysis. For example, the deterioration detection model can be trained to provide a score such as a likelihood that the patient is experiencing deterioration, based on the analysis of the decomposed vital sign data. The score or likelihood can be a likelihood that the patient (and thus the vital sign data) is part of the negative class or part of the positive class. The score or likelihood can be any possible score, such as a probability of how much the patient or vital sign data is likely to be part of the negative class or part of the positive class. Thus, according to an embodiment, the deterioration detection model can be trained using a population-based method as described or otherwise envisioned herein. For example, the data of negative patients (i.e., who did not experience deterioration) are used to train the deterioration detection model, which can optionally be a One Class Classification (OCC) model.

According to another embodiment, the deterioration detection model can be trained using a patient-based method as described or otherwise envisioned herein. For example, the patient-based method can use previous data of the same patient to train the model and to predict its own data. An example is shown FIG. 5. In this example, the test set consist of 12 hours (test time window), which results in 12 predictions. There are two decision-moments per day (7 PM (“19 u”) and 7 AM (“7 u”)). For each decision-moment, the decision to give an alarm or not depends on the highest abnormality score of the test time window. The gray bar 510 represents a decision moment for the system. Of course, the patient-based model training is not limited to this example and thus many variations are possible.

According to an embodiment, the model output can be similar for both population-based and patient-based model training. Depending on the exact method that is used, the output can be a probability (between 0 and 1) of a patient being abnormal (and thus risk of deterioration). It could also be that the model output is a number that is difficult to interpret and/or has no unit. If the outcome is a probability or another continuous number, a threshold should be chosen that converts the model output to the decision to give an alarm or not. The threshold can be chosen according to the trade-off between sensitivity and specificity. For example, a strict threshold will only detect a few patients with high risk of deterioration (low sensitivity) but has only a few false alarms (high specificity). In contrast, a lenient threshold will detect all patients who truly have a high risk of deterioration (high sensitivity) but also gives many false alarms (low specificity). This is a common trade-off, in which the threshold choice depends on patient population, hospital policy, and bed occupation, among others. Note that the model output could also be converted to different risk levels, for example: green (no risk), yellow (medium risk) or red (high risk). The model outcome could also be binary, thus indicating risk of deterioration or no risk of deterioration. The type of model outcome depends on the training method to use.

Thus, following training the deterioration detection model is a specialized model configured to receive the specialized input (namely, the one or more components and/or the raw vital sign data) and generate the very specific output, namely an indication of whether the patient is experiencing or is not experiencing deterioration, or whether the patient is facing an upcoming or impending deterioration.

At step 430 of the method, the trained deterioration detection model is stored for future use. According to an embodiment, the trained deterioration detection model may be stored in local or remote storage.

Returning to method 100 in FIG. 1, at step 160 of the method, the deterioration detection system 200 provides the determine or identification of the deterioration in the patient's condition to a user, such as a patient or clinician, via a user interface. The provided output can be any of the information as described or otherwise envisioned herein, including but not limited to the score described in detail above. The system may provide the information to a user via any mechanism, including but not limited to a visual display, an audible notification, a page, or any other method of notification. The information may be communicated by wired and/or wireless communication to another device. For example, the system may communicate the information to a monitor, screen, mobile phone, computer, laptop, wearable device, and/or any other device configured to allow display and/or other communication of the information.

In accordance with another embodiment, the deterioration detection model or system can be configured to include other information as the output, alternative to or in addition to a score or other identification of a deterioration. For example, the reporting may be or include a warning or another follow-up action (e.g., calling the patient), such as when the score exceeds a properly chosen threshold. Notably, the comparing and warning may be done each time a score is determined (e.g., each hour), or just a few times a day (e.g., twice a day) by looking back on the score during the preceding hours and checking if or how often the threshold was exceeded.

At optional step 170 of the method, the system can obtain, during a second time period after the first time period, additional vital sign data. This vital sign data or signal can be any of the vital sign data described or otherwise envisioned herein, such as body temperature, pulse or heart rate, respiration rate, blood pressure, activity, and posture, among other possible vital sign data. The deterioration detection system 200 can receive the vital sign signal from the patient in real-time, or can receive it periodically or in response to a request for data. For example, the vital sign sensor, wearable device, and/or deterioration detection system can be designed or programmed to provide vital sign data in real-time, periodically pursuant to a predetermined schedule or trigger(s), and/or in response to a request for vital sign data from a user, a clinician, and/or from another component of the deterioration detection system. According to another embodiment, some or all of the vital sign data is received from the EMR database 280. Once received, the vital sign signal may be utilized immediately and/or may be stored in local and/or remote memory.

According to an embodiment, the detection can be carried out repeatedly, e.g. every hour, based on all available data up to then. Upon the next hour, there will be one hour of extra data to take into consideration, so one or more of the components can be extended by that time interval. Subsequently, warnings may be issued every time a new assessment is done (e.g., each hour in this case), but alternatively it may be done only a few times a day, for instance every 12 hours, taking all new assessments into account that were determined during the last 12 hours. Many other options are possible.

Once the additional vital sign data is received, the system can proceed to analyze the vital sign data as described or otherwise envisioned herein. For example, the system can return to step 130 in order to decompose the additional vital sign data into the one or more components, comprising: (i) the trend component characterizing a mean of the vital sign data over the second time period: (ii) the periodic component characterizing periodicity of the vital sign data over the second time period; and (iii) the residual component over the second time period. Then at steps 140 and 150, the system can analyze the decomposed component(s) to determine whether there is deterioration, and/or to identify deterioration. That deterioration can then be reported at step 160.

Referring to FIG. 2 is a schematic representation of a deterioration detection system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a central processing unit (CPU), graphical processing unit (GPU), tensor processing unit (TPU), neural processing unit (NPU), microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may be located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, hard disk drive (HDD), solid state drive (SSD), flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, system 200 comprises or is in direct or indirect communication with a wearable device 270. The wearable device is any device that is in direct or indirect communication with a patient 274. For example, the wearable device may be a device designed to be worn by the patient, such as on the arm, around the neck, on or around the face, or anywhere else on the patient's body. The wearable device may be clipped or connected to the patient's clothing. The wearable device may be in proximity to the patient without direct contact. For example, the wearable device may be near the patient and able to obtain vital sign data via a mechanism such as a camera, sound sensor, and/or via any other mechanism for obtaining vital sign information. Many other forms of the wearable device are possible. According to an embodiment, the wearable device 270 comprises a vital sign sensor 272. The vital sign sensor is any sensor configured or otherwise capable of obtaining a vital sign signal from the patient 274. The vital sign may be any vital sign obtainable from a patient, including but not limited to body temperature, pulse or heart rate, respiration rate, blood pressure, and more.

According to an embodiment, system 200 comprises or is in direct or indirect communication with an electronic medical record system and/or an electronic medical records (EMR) database 280 from which the information about patients, including demographic, diagnosis, and/or treatment information, may be obtained or received. For example, EMR database may comprise historical vital sign data about the patient, among other patient information. According to an embodiment, the electronic medical record system 280 may be a local or remote database and is in direct and/or indirect communication with system 200. Thus, according to an embodiment, the system comprises an electronic medical record database or system 280.

According to an embodiment, storage 260 of system 200 may store one or more algorithms, modules, and/or instructions to carry out one or more functions or steps of the methods described or otherwise envisioned herein. For example, storage 260 may comprise, among other instructions or data, vital sign data 262, decomposition instructions 263, a trained deterioration detection model 264, training instructions 265, and/or reporting instructions 266.

According to an embodiment, vital sign data 262 comprises vital sign data from patient 274 via the vital sign sensor 272 of wearable device 270. This vital sign data or signal can be any of the vital sign data described or otherwise envisioned herein, such as body temperature, pulse or heart rate, respiration rate, and blood pressure, among other possible vital sign data. The deterioration detection system 200 can receive the vital sign signal from the patient in real-time, or can receive it periodically or in response to a request for data. For example, the vital sign sensor, wearable device, and/or deterioration detection system can be designed or programmed to provide vital sign data in real-time, periodically pursuant to a predetermined schedule or trigger(s), and/or in response to a request for vital sign data from a user, a clinician, and/or from another component of the deterioration detection system. According to another embodiment, some or all of the vital sign data is received from the EMR database 280. Once received, the vital sign signal may be utilized immediately and/or may be stored in local and/or remote memory.

According to an embodiment, decomposition instructions 263 direct the system to decompose vital sign data into one or more components. The first component is a trend component, which describes how the mean of the signal behaves over time. The second component is a periodic or seasonality component, which describes the periodicity of the time series, which for vital signs is typically a 24-hour circadian rhythm although the time can vary. The third component is a residual component that exists due to random noise or an unknown cause, which may be an unnoticed deterioration. One advantage of this decomposition approach is that it yields or characterizes information on the circadian rhythm that may be relevant for deterioration detection, and also yields or characterizes other variations when they are placed into a more appropriate context. Once generated, the one or more components may be utilized immediately and/or may be stored in local and/or remote memory.

According to an embodiment, trained deterioration detection model 264 of the deterioration detection system 200 is trained to analyze the decomposed component(s) (optionally including the raw data) to determine whether the patient is experiencing deterioration. The trained deterioration detection model can be any model that can be trained to utilize the input to generate the output, as described or otherwise envisioned herein. For example, the trained deterioration detection model can be a neural network or other trained machine learning model. Thus, according to an embodiment, the deterioration detection system comprises a trained deterioration detection model that receives the input data and outputs a score or other indication of deterioration when that deterioration is detected. The trained deterioration detection model is unique based on the training data used to train the model. Once generated, the trained deterioration detection model 264 can be utilized immediately, or it may be stored in local and/or remote memory for future use.

According to an embodiment, training instructions 265 direct the system to train a deterioration detection model of the deterioration detection system 200. The instructions direct the system to retrieve, obtain, or receive training data. The training data can be any data sufficient to train the model to utilize the described input data to generate the described output. For example, the training data may comprise decomposed components and/or raw data for a plurality of patients, including with ground truth optimization. The training instructions 263 further direct the system to train the deterioration detection model using the obtained training data. The deterioration detection model can be trained using a variety of different training methods. The training instructions 263 further direct the system to store the trained deterioration detection model for future use.

According to an embodiment, the deterioration detection system 200 is configured to process many thousands or millions of datapoints in the input data used to train the deterioration detection model, such as via the training instructions. For example, generating a functional and skilled trained deterioration detection model from a corpus of training data requires processing of millions of datapoints from input data and generated features. This can require millions or billions of calculations to generate a novel trained deterioration detection model from those millions of datapoints and millions or billions of calculations. As a result, each trained deterioration detection model is novel and distinct based on the input data and parameters of the model, and thus improves the functioning of the system. Generating a functional and skilled trained deterioration detection model comprises a process with a volume of calculation and analysis that a human brain cannot accomplish in a lifetime, or multiple lifetimes.

According to an embodiment, reporting instructions 266 direct the system to provide the output of the system to a user, such as a patient or clinician, via a user interface. The provided output can be any of the information as described or otherwise envisioned herein, including but not limited to the indication of deterioration and/or a deterioration score. The system may provide the information to a user via any mechanism, including but not limited to a visual display, an audible notification, a page, or any other method of notification. The information may be communicated by wired and/or wireless communication to another device. For example, the system may communicate the information to a monitor, screen, mobile phone, computer, laptop, wearable device, and/or any other device configured to allow display and/or other communication of the information.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used.

Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for identifying deterioration of a patient's condition, comprising:
obtaining, by a wearable vital sign sensor from the patient during a first time period, vital sign data:
decomposing the vital sign data into one or more components, comprising one or more of: (i) a trend component characterizing a mean of the vital sign data over the first time period; (ii) a periodic component characterizing periodicity of the vital sign data over the first time period; and (iii) a residual component over the first time period:
analyzing the one or more components to determine whether there is deterioration in the patient's condition:
identifying, based on the analysis, a deterioration in the patient's condition, wherein the deterioration is a current deterioration or an impending deterioration; and
reporting the identification of the deterioration in the patient's condition.

2. The method of claim 1, further comprising:
obtaining, for a second time period after the first time period, additional vital sign data:
decomposing the additional vital sign data into one or more components, comprising: (i) the trend component characterizing a mean of the vital sign data over the second time period; (ii) the periodic component characterizing periodicity of the vital sign data over the second time period; and (iii) the residual component over the second time period;
wherein the one or more components over the second time period are analyzed to determine whether there is deterioration in the patient's condition.

3. The method of claim 1, further comprising the step of transmitting the vital sign data to a remote server.

4. The method of claim 1, wherein the residual component characterizes variation in the vital sign data, wherein the variation is not explained by the trend component or the periodic component.

5. The method of claim 1, wherein decomposing the vital sign data into the one or more components comprises inflation weighting.

6. The method of claim 1, wherein the first time period is at least 24 hours, or at least one circadian rhythm.

7. The method of claim 1, wherein the vital sign data is one or more of heart rate, blood pressure, respiration rate, activity, and posture.

8. The method of claim 1, wherein one or more of the steps of: (i) analyzing the one or more components to determine whether there is deterioration in the patient's condition; and (ii) identifying, based on the analysis, deterioration in the patient's condition, comprises a machine learning algorithm trained to perform the analysis and/or identification.

9. The method of claim 1, wherein identifying, based on the analysis, deterioration in the patient's condition comprises a deterioration score, and wherein reporting the identification of the deterioration in the patient's condition comprises the deterioration score.

10. A system for identifying deterioration of a patient's condition, comprising:
a wearable device comprising a vital sign sensor configured to obtain vital sign data from a patient:
a processor configured to: (i) decompose the vital sign data into one or more components, comprising: (1) a trend component characterizing a mean of the vital sign data over the first time period: (2) a periodic component characterizing periodicity of the vital sign data over the first time period; and (3) a residual component over the first time period: (ii) analyze the one or more components to determine whether there is deterioration in the patient's condition: (iii) identify, based on the analysis, a deterioration in the patient's condition, wherein the deterioration is a current deterioration or an impending deterioration; and
a user interface configured to report the identification of the deterioration in the patient's condition.

11. The system of claim 10, wherein decomposing the vital sign data into at least three components comprises inflation weighting.

12. The system of claim 10, wherein the first time period is at least 24 hours, or at least one circadian rhythm.

13. The system of claim 10, wherein the vital sign data is one or more of heart rate, blood pressure, respiration rate, activity, and posture.

14. The system of claim 10, wherein the system further comprises a trained deterioration detection model, and wherein one or more of: (i) analyzing the one or more components to determine whether there is deterioration in the patient's condition; and (ii) identifying, based on the analysis, deterioration in the patient's condition, comprises use of the trained deterioration detection model.

15. The system of claim 10, wherein identifying, based on the analysis, deterioration in the patient's condition comprises a deterioration score, and wherein reporting the identification of the deterioration in the patient's condition comprises the deterioration score.
